(19) United States
(12) Patent Application Publication
(10) Pub. No.: US 2009/0215160 A1
(43) Pub. Date: Aug. 27, 2009

(54) ENDOSCOPE WASHING AND DISINFECTING EVALUATION APPARATUS

(75) Inventors: Tsuruo Hatori, Sagamihara-shi (JP); Yuki Nagai, Tokyo (JP); Toshiharu Kinoshita, Tokyo (JP); Hidetaka Tsuji, Tokyo (JP); Yosuke Kanamori, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/365,938

(22) Filed: Feb. 5, 2009

(30) Foreign Application Priority Data
Feb. 22, 2008 (JP) 2008-041676

(51) Int. Cl. C12M 1/34

(52) U.S. Cl. 435/287.4

(57) ABSTRACT

A connection base portion of a stripping solution unit storing a stripping solution is connected to an insertion opening of a channel in an insertion portion of an endoscope. A distal end portion of the insertion portion formed with a distal end opening of the channel is attached, with a sealing film, to an opening in a top cover of a bacteria collection container with a filter inside to be sealed from outside. By suction operation of a syringe at a bottom of the bacteria collection container, the stripping solution is sucked into the insertion opening, flows out from the distal end opening through the channel, and is filtered through the filter. A medium is supplied to the filter for cultivation. A result of the cultivation is observed, and an evaluation result as to cleanliness is obtained from presence or absence of bacteria.

This application claims benefit of Japanese Application No. 2008-041676 filed in Japan on Feb. 22, 2008; the contents of which are incorporated by this reference.

### BACKGROUND OF THE INVENTION

1. Field of the Invention

The present invention relates to an endoscope washing and disinfecting evaluation apparatus for evaluating quality of endoscope washing and disinfecting by a washing and disinfecting apparatus.

2. Description of the Related Art

In recent years, endoscopes have been in wide use in a field of medicine and in industrial fields. Ordinary endoscopes are repeatedly used in endoscopic examinations. An endoscope after being used in an endoscopic examination is subjected to washing and disinfecting processing by a washing and disinfecting apparatus.

The endoscope which has been cleaned by the washing and disinfecting processing is to be used in a next endoscopic examination.

As described above, endoscopes are subjected to washing and disinfecting processing by a washing and disinfecting apparatus. An examination for evaluating, in particular, quality of washing and disinfecting (i.e., cleanliness) of a channel of an endoscope is desired to be performed on an endoscope which has been subjected to washing and disinfecting processing at appropriate intervals to check or evaluate quality of the washing and disinfecting processing.

In a conventional method, when cleanliness of a channel of an endoscope is to be evaluated, sterile water is infused from one opening into the channel by a syringe, a distal end portion of the endoscope where the channel opens is put into a collection container such as a beaker, and the sterile water having passed through the channel is collected as a collection liquid by the collection container.

After the collection, the collection liquid is filtered through a filter which traps only bacteria, and bacteria adherent to the filter are cultivated using a medium. The cleanliness is evaluated from a result of the cultivation using the medium.

Japanese Patent Application Laid-Open Publication No. 2006-326225 discloses an evaluation apparatus including a hollow body simulating an endoscope with a channel, a first housing portion in which a biological indicator for performing sterilization evaluation on the hollow body is housed, and a second housing portion in which a medium solution for cultivating bacteria adherent to the biological indicator is housed.

The evaluation apparatus is subjected to sterilization processing by a single sterilization apparatus together with an endoscope. With the process, sterilization of an endoscope by the sterilization apparatus is evaluated without using an actual endoscope.

Note that Japanese Patent Application Laid-Open Publication No. 11-133030 discloses a detection method including attaching an acid pigment solution to an article surface to which protein may be fixed, removing the acid pigment solution from the article surface by cleaning, bringing a predetermined amount of an alkali extraction liquid into contact with the article surface after being cleaned, collecting the extraction liquid, and detecting presence or absence of protein fixation to the article surface and the degree of fixation from acid pigments dissolved in the extraction liquid.

### SUMMARY OF THE INVENTION

An endoscope washing and disinfecting evaluation apparatus according to the present invention includes a stripping solution storage section attached, in a state substantially sealed from outside, to one opening of a channel of an endoscope for storing a stripping solution to be infused from the one opening into the channel, a bacteria collection container attached, in a state substantially sealed from outside, to the other opening of the channel in which a filter for trapping a bacterium contained in the stripping solution when the stripping solution having passed through the channel and flowed out from the other opening is filtered through the filter is placed, and a medium supply section for placing the bacterium trapped by the filter in a state substantially sealed from outside and supplying a medium for cultivation.

### BRIEF DESCRIPTION OF THE DRAWINGS

FIG. 1 is a view showing an evaluation apparatus according to a first embodiment of the present invention in an example of how the evaluation apparatus is used;

FIG. 2 is a view of the evaluation apparatus in kit form;

FIG. 3 is a flow chart showing evaluation procedures;

FIG. 4 is a view showing a bacteria collection unit portion when a stripping solution is collected;

FIG. 5 is a view showing the bacteria collection unit portion when a medium solution is infused;

FIG. 6A is a view showing how the evaluation apparatus is applied to an air supply channel and a water supply channel;

FIG. 6B is a view showing how a stripping solution unit is connected to a suction base to evaluate cleanliness;

FIG. 7 is a view showing an evaluation apparatus according to a second embodiment of the present invention in an example of how the evaluation apparatus is used;

FIG. 8 is a flow chart showing evaluation procedures;

FIG. 9A is a view showing a configuration of a bacteria collection unit according to a first modification of the second embodiment in an example of how the bacteria collection unit is used;

FIG. 9B is a view showing a top cover with a filter at a lower end which is removed from a bacteria collection container;

FIG. 9C is a view showing a state in which the filter at the lower end of the top cover in FIG. 9B is pushed against a medium in a medium container;

FIG. 9D is a view showing a state, after the state in FIG. 9C, in which an upper surface of the medium container with the filter left is covered with a top cover;

FIG. 10A is a view showing an example of a configuration in which a stripping solution having passed through a channel of an endoscope is collected, according to a second modification of the second embodiment;

FIG. 10B is a view showing an example of a configuration in which a collection container into which the stripping solution is collected is attached to a bacteria collection unit, and bacteria are trapped by the filter;

FIG. 11 is a view showing a configuration of an evaluation apparatus according to a third embodiment of the present invention;

FIG. 12 is a view showing the evaluation apparatus according to the third embodiment of the present invention in an example of how the evaluation apparatus is used;

FIG. 13 is a flow chart showing evaluation procedures;

FIG. 14A is a view showing an example of a configuration of a peripheral portion of a container which collects a stripping solution, according to a modification of the third embodiment;

FIG. 14B is a view showing a modification of a brush using a cotton portion according to the third embodiment;

FIG. 15 is a view showing a configuration of a cleanliness evaluation apparatus according to a fourth embodiment of the present invention;

FIG. 16 is a flow chart showing evaluation procedures;

FIG. 17A is a view showing a filter main body when a cover film is removed;

FIG. 17B is a view showing a state in which the filter main body in FIG. 17A is pushed against an outer surface of an insertion portion of an endoscope;

FIG. 17C is a view showing how a tube is connected to the filter main body after the operation in FIG. 17B, and a medium solution is infused into the filter main body;

FIG. 17D is a view showing a state in which the filter main body after the operation in FIG. 17C is stored in a Petri dish;

FIG. 18A is a perspective view showing a configuration of a filter main body according to a first modification of the fourth embodiment;

FIG. 18B is a sectional view of FIG. 18A;

FIG. 19 is a perspective view showing a configuration of an evaluation apparatus main body according to a second modification of the fourth embodiment; and

FIG. 20 is a view showing how a distal end surface of the insertion portion of the endoscope is pushed against a solid medium of the evaluation apparatus main body in FIG. 19.

### DETAILED DESCRIPTION OF THE PREFERRED EMBODIMENTS

Embodiments of the present invention will be described below with reference to the drawings.

### First Embodiment

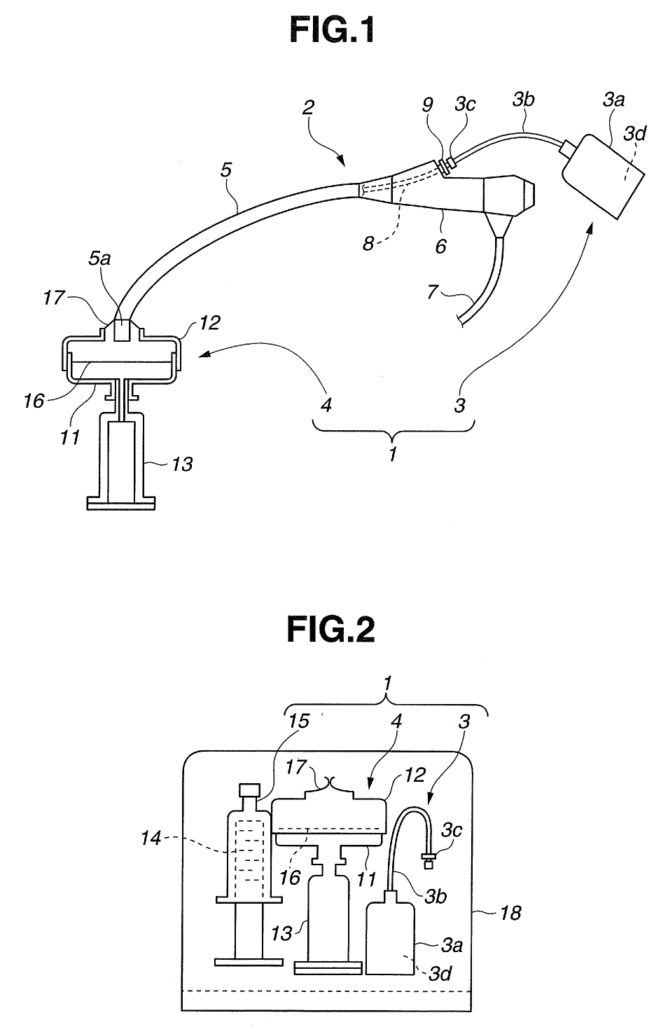

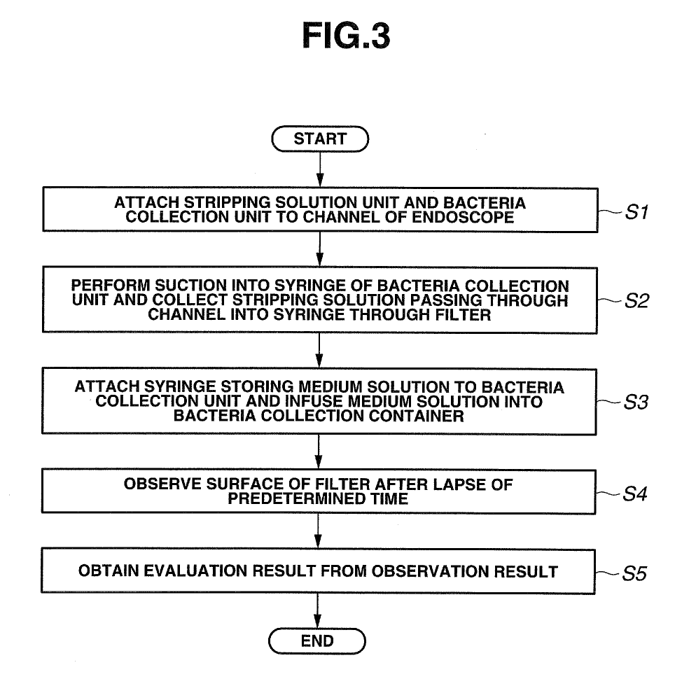

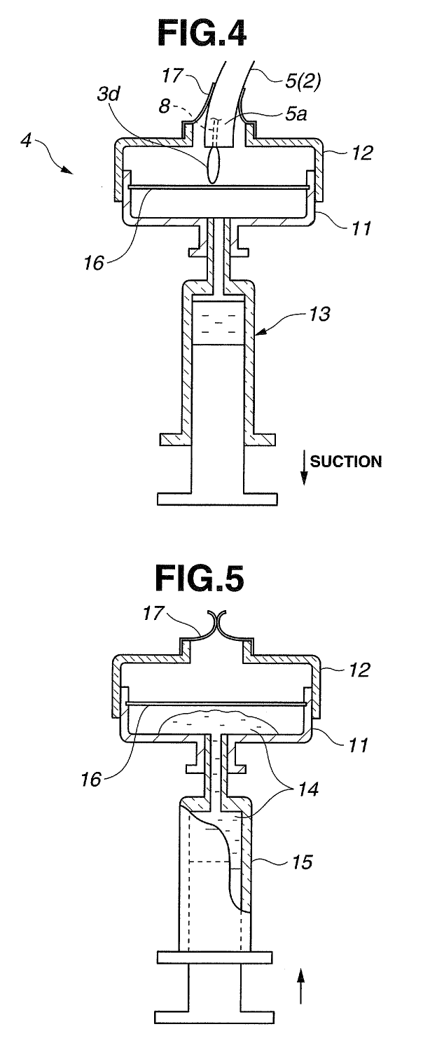

FIGS. 1 to 6B relate to a first embodiment of the present invention. FIG. 1 shows an endoscope washing and disinfecting evaluation apparatus according to the first embodiment in an example of how the endoscope washing and disinfecting evaluation apparatus is used. FIG. 2 shows the endoscope washing and disinfecting evaluation apparatus in kit form. FIG. 3 shows evaluation procedures. FIG. 4 shows a bacteria collection unit portion when a stripping solution is collected.

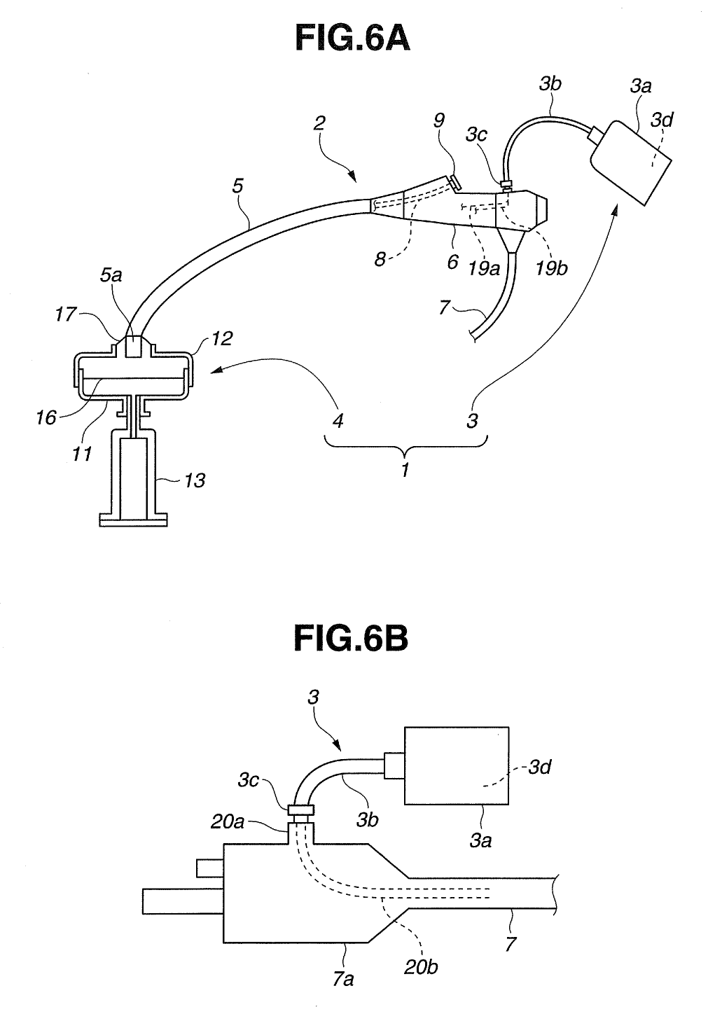

FIG. 5 shows the bacteria collection unit portion when a medium solution is infused. FIG. 6A shows how the endoscope washing and disinfecting evaluation apparatus is applied to an air supply channel and a water supply channel. FIG. 6B shows how the endoscope washing and disinfecting evaluation apparatus is applied to a suction line.

As shown in FIG. 1, an endoscope washing and disinfecting evaluation apparatus (hereinafter simply abbreviated as an evaluation apparatus) 1 according to the first embodiment of the present invention includes a stripping solution unit 3 which is detachably attached to one end portion of a channel serving as a target at the time of evaluating washing and disinfecting of an endoscope 2, and a bacteria collection unit 4 which is attached to the other end portion.

The endoscope 2 has an elongated insertion portion 5 which is inserted into a body cavity for an endoscopic examination, an operation portion 6 which is provided at a rear end of the insertion portion 5, and a universal cable 7 extending from the operation portion 6.

A channel 8 called a treatment instrument channel into which a treatment instrument (not shown) is to be inserted is provided in the insertion portion 5. A distal end of the channel 8 opens at a distal end surface of the insertion portion 5 while a rear end of the channel 8 opens at a treatment instrument insertion opening (hereinafter abbreviated as an insertion opening) 9 near a front end of the operation portion 6 (which is plugged when not in use).

The endoscope 2 after use in an endoscopic examination is washed and disinfected or sterilized (hereinafter the term washing and disinfecting includes sterilization) by a washing and disinfecting apparatus or sterilizing apparatus.

To evaluate or check whether the endoscope 2 after washing and disinfecting is properly washed and disinfected, the evaluation apparatus 1 is connected (attached) to the endoscope 2, as shown in FIG. 1.

In the stripping solution unit 3, a clean stripping solution 3d is stored in a sealed container 3a. One end of a tube 3b is fixed to a cap at a distal end of the container 3a while a connection base portion 3c which is detachably connected to one end of the channel 8 serving as the target at the time of evaluating washing and disinfecting (i.e., cleanliness) of the endoscope 2 is provided at the other end of the tube 3b.

Specifically, the one end of the channel 8 in the example of FIG. 1 refers to the insertion opening 9. The connection base portion 3c is detachably connected to the insertion opening 9 by, e.g., press fitting such that the connection base portion 3c is sealed from outside.

Note that a configuration in which the tube 3b is not extended, the connection base portion 3c is provided at the stripping solution unit 3, and the stripping solution unit 3 is connected to the insertion opening 9 may be adopted. The container 3a storing the stripping solution in the stripping solution unit 3 is formed of a flexible container which deforms under pressure or the like.

The bacteria collection unit 4 is detachably connected to a distal end portion 5a of the insertion portion 5 where the other end of the channel 8 opens.

The bacteria collection unit 4 includes a bacteria collection container 11 in, e.g., an annular shape in which a cavity is formed, a top cover 12 which covers an open upper end of the bacteria collection container 11, a syringe 13 detachably connected to an open base at a bottom of the bacteria collection container 11 and serving as a suction section or a collection section for collecting the stripping solution 3d through the channel 8 by suction operation, and a syringe 15 (see FIGS. 2 and 5) for infusing a medium solution 14 (to be described later).

In the bacteria collection container 11, a circumferential groove is formed at an appropriate height in an inner peripheral surface, and a filter 16 having a large number of minute openings which allows passage of the stripping solution 3d but does not allow passage of bacteria and traps the bacteria is attached. The filter 16 is detachable from the circumferential groove.

The medium solution 14 is supplied to the filter 16 having trapped bacteria through infusion by the syringe 15 serving as a medium supply section.

A base projecting upward is provided, e.g., near a center of the top cover 12, and a sealing film 17 which covers an opening in the top cover 12 is attached to the base.

The sealing film 17 has, for example, an annular portion which adheres tightly to the base and an openable portion on an upper end side of the annular portion which is projecting in, e.g., a conical shape, has a cross-shaped cut at a center, and can open and close freely by virtue of the cut.

Under normal conditions, elastic force of an elastic member forming the sealing film 17 brings segments of a cut portion into contact with each other and maintains a sealed state. Pushing the openable portion with the distal end portion 5a of the endoscope 2 opens the cut and lets the distal end portion 5a in. In this case, the cut portion adheres tightly to an outer peripheral surface of the distal end portion 5a and maintains a state sealed from outside.

Note that distal end portions of the segments of the cut portion typically expand, as shown in, e.g., FIG. 5. However, the cut portion is not limited to the shape. The cut portion may be configured to be deformed such that the distal end portions of the segments of the cut portion expand inward when the cut portion is pushed by the distal end portion 5a.

Alternatively, in order to improve sealing function, magnet powder or the like may be mixed in the cut portion, and the openable portion may be configured (to improve the sealing function) such that adjacent ones of the segments of the cut portion stick to each other due to magnetic force when the cut portion is not pushed or when the distal end portion 5a is inserted.

The present invention is not limited to the openable portion with the above-described structure. An opening portion through which the distal end portion 5a passes in close contact by press fitting or the like may be formed at the center of the sealing film 17, and the opening portion may be freely opened and closed with a cover. Alternatively, an opening portion of the base of the top cover 12 may be formed with an elastic member having an inner diameter slightly smaller than an outer diameter of the distal end portion 5a such that the distal end portion 5a can be press-fitted into the opening portion of the top cover 12, and the opening portion may be closed with a cover or the like when the distal end portion 5a is not inserted.

Note that the evaluation apparatus 1 before use is stored in a sterilized transparent bag 18 made of plastic or the like, as shown in, e.g., FIG. 2, and is prepared as an evaluation kit including all members required for evaluation. The evaluation apparatus 1 is configured to be capable of easily evaluating whether bacteria are left in the channel of the endoscope 2, i.e., cleanliness after being taken out from the bag 18.

Note that, as an example, the stripping solution unit 3 filled with the stripping solution 3d, the bacteria collection unit 4 with the syringe 13 attached to the bacteria collection container 11, and the syringe 15 infused with the medium solution 14 are stored in the bag 18.

In the stripping solution unit 3, the connection base portion 3c is hermetically sealed with sealing tape or the like. In the bacteria collection unit 4, a connection between the bacteria collection container 11 and the syringe 13 is hermetically sealed with sealing tap or the like and is fixed (so as not to move).

In the syringe 15, a distal end opening portion is hermetically sealed with sealing tape or the like, and a syringe cylindrical container which is infused with the medium solution 14 and a movable portion which can slide inside the syringe cylindrical container are fixed to each other with sealing tape or the like.

The work of setting up the evaluation apparatus 1 to be ready for evaluation can be easily performed by removing sealing tape or the like.

Note that the present invention is not limited to the example shown in FIG. 2. For example, the syringe 13 may be separated from the bacteria collection unit 4 not to be attached, and the medium solution 14 may be stored in a container different from the syringe 15.

A method for evaluating cleanliness (related to washing and disinfecting processing) of a channel of the endoscope 2 will be described with reference to FIG. 3. Although an example in which cleanliness of the channel 8 as the treatment instrument channel is evaluated will be described here, the same applies to an air supply channel and a water supply channel.

A user takes out the endoscope 2 to be subjected to cleanliness evaluation from a repository or the like which has been washed and disinfected. The user also takes out the stripping solution unit 3 and bacteria collection unit 4 from the bag 18.

The user attaches the stripping solution unit 3 and bacteria collection unit 4 to the one and other ends, respectively, of the channel 8 (serving as an evaluation target), as shown in step S1 in FIG. 3.

More specifically, the user attaches the stripping solution unit 3 to the insertion opening 9 and attaches the bacteria collection unit 4 to the distal end portion 5a (or attaches the distal end portion 5a to the bacteria collection unit 4).

A state after the attachment is as shown in FIG. 1. A portion where the stripping solution unit 3 is attached to the insertion opening 9, and a portion where the bacteria collection unit 4 is attached to the distal end portion 5a are to be evaluated in a sealed or closed system, not an open system. For this reason, it is possible to eliminate influence of contamination by bacteria and the like in the air in an open system.

The user operates a movable portion of the syringe 13 attached to the bacteria collection unit 4, as shown in step S2. That is, the user performs suction operation such that a sucking negative pressure space is formed inside the syringe 13. With the suction operation, the stripping solution 3d in the stripping solution unit 3 is sucked toward the insertion opening 9 and passes from the insertion opening 9 through the channel 8.

Note that, in the suction operation, the container 3a of the stripping solution unit 3 may be deformed to facilitate infusion of the stripping solution 3d in the container 3a from the insertion opening 9 into the channel 8.

The stripping solution 3d having been taken from a distal end opening of the channel 8 at the distal end portion 5a into the bacteria collection unit 4, to which the distal end opening is attached in a state sealed from outside, is passed through (i.e., is filtered through) the filter 16 placed in the bacteria collection container 11 and is collected into the syringe 13.

FIG. 4 shows the collection. As described above, the stripping solution 3d, which passes through the channel 8 and strips deposit from an inner surface of the channel 8, is filtered through the filter 16 placed in the bacteria collection container 11 by suction operation of the syringe 13. With the filtering, bacteria contained in the stripping solution 3d are trapped by the filter 16 and are left on an upper surface of the filter 16.

After the user performs suction into the syringe 13 and collects the stripping solution 3d into the syringe 13 by an amount set in advance, the user removes the distal end portion 5a of the endoscope 2 from the bacteria collection unit 4. When the distal end portion 5a of the endoscope 2 is removed from the bacteria collection unit 4, the base portion at an upper end of the bacteria collection unit 4 is sealed by the openable portion at the sealing film 17.

The user also removes the syringe 13 after the use in the collection from the bacteria collection container 11.

The user takes out the syringe 15 infused with the medium solution 14 from the bag 18 and attaches an inlet of the syringe 15 to the bacteria collection container 11, as shown in step S3.

The user operates the movable portion of the syringe 15 and infuses the medium solution 14 stored in the syringe 15 into the bacteria collection container 11. FIG. 5 shows how the syringe 15 is attached, and the medium solution 14 is infused into the bacteria collection container 11.

The amount of the medium solution 14 when the medium solution 14 reaches the filter 16 serves as a guide for determining a predetermined amount by which the medium solution 14 is to be infused. Note that a state in FIG. 5 is a state before the medium solution 14 reaches the filter 16, and the operation of further pushing forward the movable portion of the syringe 15 suffices to make the medium solution 14 reach the filter 16.

A calibration marking corresponding to the predetermined amount of the syringe 15 may be marked to allow the user to achieve a state in which the medium solution 14 has reached the filter 16 by pushing forward the movable portion (piston) of the syringe 15 to the marked calibration marking. Alternatively, the bacteria collection unit 4 may be configured such that the medium solution 14 reaches the filter 16 when the user pushes forward the movable portion to an upper limit of movable scope.

The bacteria collection container 11 may be formed of an optically transparent member to allow the user to check how the medium solution 14 is infused.

Note that although the base at the bottom of the bacteria collection container 11 is open to outside from when the syringe 13 is removed from the bacteria collection container 11 to when the syringe 15 is attached, quick replacement of the syringe 13 with the syringe 15 allows a sufficient reduction in contamination by bacteria within the time for replacement. That is, it is possible for the bacteria collection container 11 to maintain a substantially-sealed-system state.

An openable portion like the openable portion of the above sealing film 17 may be attached to the base portion in order to reduce or eliminate contamination by bacteria.

Note that if the medium solution 14 spills out when the syringe 15 is removed from the bacteria collection container 11 after step S3, a sealing film may be attached as described above to also function to prevent an outflow.

After the user infuses the medium solution 14 into the bacteria collection container 11 by the predetermined amount, as in step S3, the user performs the operation of placing the bacteria collection container 11 at an appropriate location or the like to cultivate or propagate in the medium solution bacteria trapped by the filter 16, if any, as shown in step S4.

The user observes a surface of the filter 16 of the bacteria collection container 11 after a lapse of a predetermined time.

Note that the top cover 12 is made of, e.g., an optically transparent member, and the surface of the filter 16 can be observed without removing the top cover 12.

The user can evaluate quality of washing and disinfecting processing, i.e., cleanliness by the observation of the surface of the filter 16, as shown in step S5.

More specifically, if propagation of bacteria cannot be visually identified by the observation of the surface of the filter 16, an evaluation result indicating sufficient washing and disinfecting (including sterilization) processing, i.e., that a sufficient level of cleanliness is ensured can be obtained. On the other hand, if propagation of bacteria can be visually identified by the observation of the surface of the filter 16, an evaluation result indicating insufficient washing and disinfecting processing, i.e., insufficient cleanliness can be obtained.

Note that the predetermined time for cultivating or propagating bacteria in the medium solution 14 may be set in advance to an appropriate value such that an observation result itself as to whether propagation of bacteria can be visually identified which is obtained by the observation of the surface of the filter 16 corresponds to an evaluation result as to cleanliness. In the case, temperature conditions may be defined in addition to the time (for leaving the bacteria collection container 11 as it stands) for higher-accuracy evaluation.

As described above, according to the present embodiment, a user can obtain, with simple work, a more reliable evaluation result as to whether the channel 8 of the endoscope 2 after washing and disinfecting has been sufficiently disinfected, i.e., cleanliness in a substantially-sealed-system state. In the case, it is possible to easily obtain a reliable evaluation result without a clean room.

As a modification of the present embodiment, the filter 16 may be impregnated with a protein detection solution in advance such that a simple evaluation result based on a protein detection result can be obtained after step S2 in FIG. 3.

The protein detection solution may detect ATP (Adenosine TriPhosphate) of an organism.

In the above embodiment, the case of the channel 8 called a treatment instrument channel, whose one end is the insertion opening 9 and whose other end opens at the distal end portion 5a of the insertion portion 5, has been described. However, cleanliness evaluation can also be performed on another channel.

FIGS. 6A and 6B show examples of application of the first embodiment to another channel portion. The application example in FIG. 6A shows how the stripping solution unit 3 is attached to an air supply/water supply opening at the operation portion 6 from which an air supply/water supply valve has been removed, and washing and disinfecting of an air supply channel 19a and a water supply channel 19b is evaluated.

The air supply channel 19a and water supply channel 19b are formed in the insertion portion 5, meet on the distal end side of the insertion portion 5, and reach one end of a nozzle (not shown). The other end of the nozzle opens at the distal end surface of the distal end portion 5a.

By operation similar to (but different in parts related to the insertion opening 9 from) the operation described with reference to FIGS. 1 to 5, i.e., suction operation of the syringe 13, the stripping solution 3d is passed from one opening at the operation portion 6 through the air supply channel 19a and water supply channel 19b. The stripping solution 3d having flowed out from the distal end portion 5a is passed through the filter 16 in the bacteria collection unit 4. Operation (work) after collection of bacteria by the filter 16 is same as operation in the case of the channel 8, and an evaluation result as to washing and disinfecting of the air supply channel 19a and water supply channel 19b can be obtained.

As shown in FIG. 6B, the stripping solution unit 3 may be attached to, e.g., a suction base 20a provided in a connector 7a at an end of the universal cable 7, and washing and disinfecting of a suction line 20b may be evaluated.

The suction line 20b communicates with the channel 8 in the insertion opening 9 through the universal cable 7 and operation portion 6. In other words, the channel 8 forks near the rear end of the insertion portion 5 into branches, one extending toward the insertion opening 9 and the suction line 20b extending toward the operation portion 6.

Washing and disinfecting of the suction line 20b can be evaluated by operation similar to (but different in parts related to the insertion opening 9 from) the operation described with reference to FIGS. 1 to 5 (the insertion opening 9 is desirably plugged in the case).

As described above, according to the present embodiment, a reliable evaluation result as to cleanliness can be obtained with simple work.

### Second Embodiment

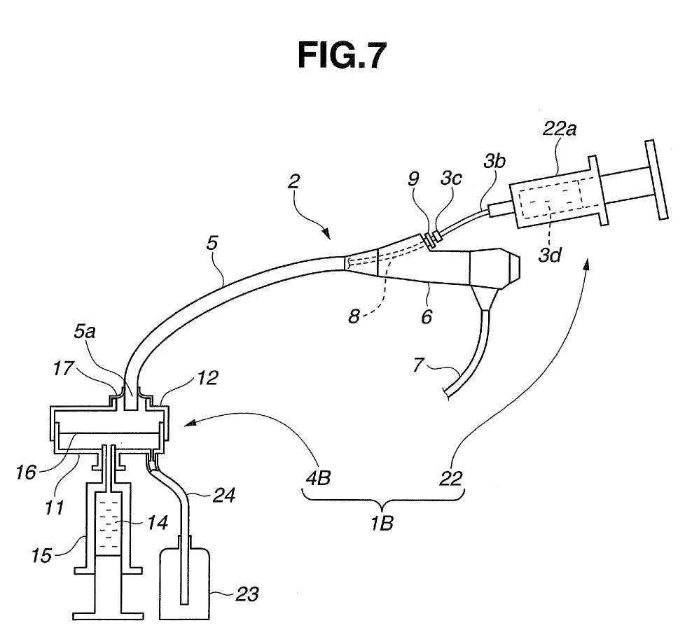

A second embodiment of the present invention will be described with reference to FIG. 7. FIG. 7 shows an evaluation apparatus 1B according to the second embodiment in an example of how the evaluation apparatus 1B is used. In the first embodiment, the stripping solution 3d is passed from one opening of the channel to the other opening by sucking the stripping solution 3d on the distal end portion 5a side of the insertion portion 5 of the endoscope 2 using the suction section. In the present embodiment, a stripping solution 3d is passed from one opening of a channel to the other opening by infusion operation of a stripping solution infusion section.

As shown in FIG. 7, the evaluation apparatus 1B according to the present embodiment is obtained by using a stripping solution unit 22 which uses a syringe 22a serving as the stripping solution infusion section storing a stripping solution 3d instead of the container 3a storing the stripping solution 3d in the stripping solution unit 3, in the evaluation apparatus 1 shown in FIGS. 1 and 2.

A user feeds the stripping solution 3d being pushed forward from the syringe 22a from an insertion opening 9 to which the syringe 22a is attached (through a connection base portion 3c) toward a channel 8 and feeds the stripping solution 3d from an opening in the channel 8 of a distal end portion 5a which is the other end of the channel 8 toward a bacteria collection unit 4B, by performing the operation of pushing forward a movable portion of the syringe 22a.

Note that the stripping solution infusion section is not limited to the syringe 22a. For example, the container 3a according to the first embodiment may be made of a flexible member, and a user may infuse the stripping solution 3d inside from the insertion opening 9 as the one opening into the channel 8 by performing the operation of denting the container 3a.

The bacteria collection unit 4B according to the present embodiment is obtained by attaching a syringe 15 storing a medium solution 14 to a bottom of a bacteria collection container 11 instead of the syringe 13, in the bacteria collection unit 4 according to the first embodiment.

The bacteria collection container 11 is connected to a collection container 23 for collecting a stripping solution having passed through a filter 16 through, e.g., a tube 24. The present embodiment is configured not to have the syringe 13 according to the first embodiment. The present embodiment is identical in other respects to the first embodiment.

In the first embodiment, the stripping solution 3d having passed through the channel 8 and then the filter 16 is collected into the syringe 13 connected to the bottom of the bacteria collection container 11. In the present embodiment, the stripping solution 3d is collected into the collection container 23 through the tube 24.

In the present embodiment, since the syringe 15 storing the medium solution 14 is initially attached to a bottom of the bacteria collection container 11, the work of replacing the syringe 13 with the syringe 15 in the first embodiment is unnecessary. It is possible to place the bacteria collection container 11 in a state in which bacteria trapped by the filter 16 are cultivated in the medium solution 14 by operating the syringe 15 and pushing forward the medium solution 14.

That is, according to the present embodiment, the syringe replacement work is unnecessary after bacteria collection. The bacteria collection container 11 can be placed in the state in which bacteria trapped by the filter 16 are cultivated in the medium solution 14 by operating the syringe 15 and pushing forward the medium solution 14 immediately after the bacteria collection. Note that a membrane filter may be adopted as the filter.

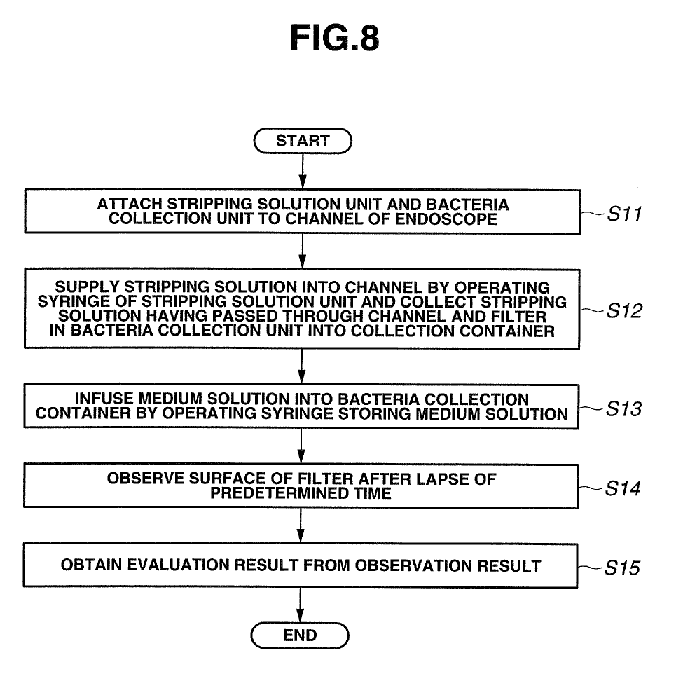

Procedures for evaluation by the evaluation apparatus 1B according to the present embodiment are as shown in FIG. 8. The procedures in FIG. 8 are similar to the procedures in FIG. 3.

As shown in a first step, S11, the stripping solution unit 22 is attached to one end of the channel 8 of the endoscope 2, and the bacteria collection unit 4B is attached to the other end.

Step S11 is same as step S1 in FIG. 3. Note that, in the present embodiment, the stripping solution unit 22 is used instead of the stripping solution unit 3, and the bacteria collection unit 4B is used instead of the bacteria collection unit 4.

In a next step, S12, a movable portion of the syringe 22a is operated, and the stripping solution 3d in the syringe 22a is pushed forward. The stripping solution 3d pushed forward is infused into the channel 8, and the infused stripping solution 3d is passed through the channel 8. The stripping solution 3d having flowed out from the distal end portion 5a into the bacteria collection unit 4B is filtered through the filter 16 and is collected by the collection container 23 through the tube 24.

In a next step, S13, the syringe 15 storing the medium solution 14 is operated, and the medium solution 14 is infused into the bacteria collection container 11. In the present embodiment, the collection container 23 for collecting the stripping solution 3d and the syringe 15 storing the medium solution 14 are separately formed, and the stripping solution 3d is collected by the collection container 23 connected to the bacteria collection container 11 through the tube 24. It is thus possible to infuse the medium solution 14 immediately after collecting the stripping solution 3d by the collection container 23.

Steps S14 and S15 after the work are same as steps S4 and S5 in FIG. 3.

According to the present embodiment, an evaluation result as to cleanliness can be obtained with simple work without the work of replacing the syringe 13 with the syringe 15 according to the first embodiment. Since replacement work is unnecessary, a sealed or closed system state can be more reliably maintained. It is thus possible to obtain a more reliable evaluation result.

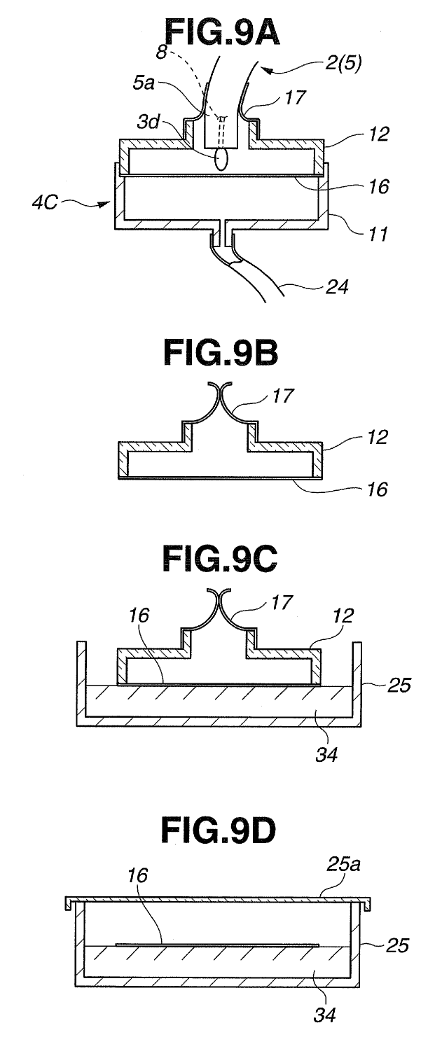

As a first modification of the present embodiment, the configuration below may be adopted. In the present modification, a bacteria collection unit 4C is adopted, as shown in FIG. 9A, instead of the bacteria collection unit 4B in FIG. 7. In the bacteria collection unit 4C, the filter 16 is attached at a lower end of the top cover 12.

Note that, in the bacteria collection unit 4C according to the present modification, the work of infusing a medium solution is unnecessary, as will be described below. Accordingly, the syringe 15 (for infusing a medium solution) in FIG. 7 is not connected to the bacteria collection container 11, and an inlet to which such a syringe is to be connected is not provided.

Although FIG. 9A shows an example of a configuration in which a stripping solution having passed through the filter 16 is collected into the collection container 23 (shown in FIG. 7) through the tube 24 connected to the bacteria collection container 11, a stripping solution may be collected into the bacteria collection container 11.

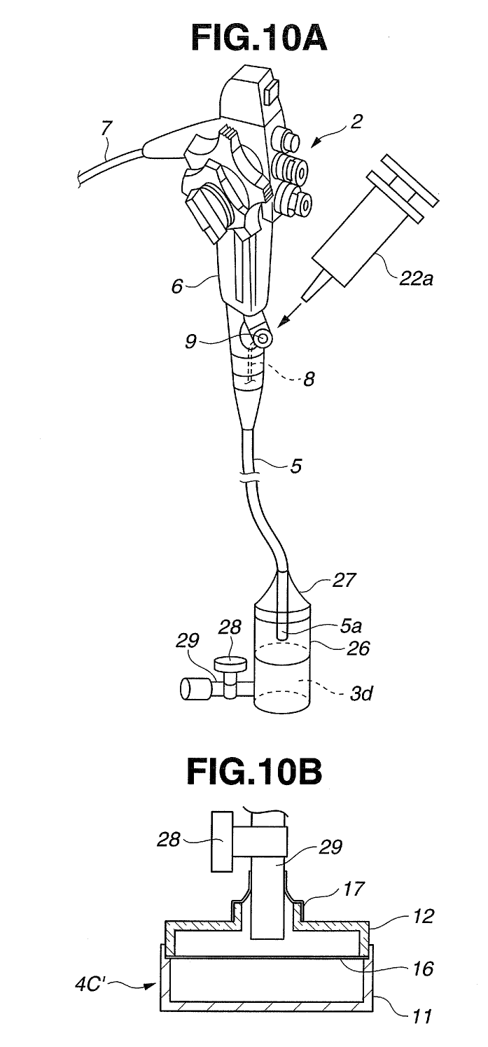

In the case of such a configuration, the tube 24 and the collection container 23 (shown in FIG. 7) are unnecessary. A base (of the bacteria collection container 11) to which the tube 24 is to be connected is also unnecessary in the case (the configuration is shown as a bacteria collection unit 4C′ in FIG. 10B (to be described later)).

After the stripping solution 3d having passed through the channel 8 is filtered through the filter 16, an endoscope 2 is removed from the bacteria collection unit 4C, and a top cover 12 to which the filter 16 is attached is removed from the bacteria collection container 11, as shown in FIG. 9B.

As shown in FIG. 9C, the filter 16 of the top cover 12 in FIG. 9B is pushed against an upper surface of a medium container 25 storing a medium 34 (or a medium solution). The filter 16 is left on the medium 34, and the upper surface of the medium container 25 is covered with a top cover 25a, as shown in FIG. 9D, to prevent bacteria from entering the medium container 25. After a lapse of a predetermined time, an observation is performed.

In the present modification, an open system state is brought about when the top cover 12 with the filter 16 attached is removed from the bacteria collection container 11 and at the time of the work of pushing the filter 16 against the medium container 25. However, a substantially-sealed-system state can be maintained by performing the work in a short time. Since the substantially-sealed-system state can be maintained, a reliable evaluation result can be obtained.

A configuration like a second modification to be described below may be adopted.

In FIG. 10A, the syringe 22a storing a stripping solution is directly connected (attached) to the insertion opening 9 of the endoscope 2. At the time of the connection, an attached portion is sealed from outside. The stripping solution is pushed forward from the syringe 22a and is infused from the insertion opening 9 into the channel 8.

The stripping solution, which has passed through the channel 8 and flowed out from a distal end of the insertion portion 5 serving as a distal end opening, is collected into a collection container 26. In this case, an open upper end of the collection container 26 is sealed with a sealing member 27 to prevent bacteria and the like from entering the collection container 26 from surroundings or the air.

Note that, in the example shown in FIG. 10A, the stripping solution 3d has been collected into the collection container 26.

For example, a base 29 including a cock 28 which opens and closes is provided near a bottom of the collection container 26. Note that a sealing cap for sealing an end of the base 29 in a clean state is attached at an end of the base 29.

A sealed system state is maintained when a stripping solution having passed through the channel 8 is collected.

In the sealed system state as in FIG. 10A, as for the stripping solution 3d collected into the collection container 26, the base 29, from which the sealing cap has been removed, is inserted into, e.g., an opening at an upper portion of the bacteria collection unit 4C′, which is a modification of the bacteria collection unit 4C described with reference to FIG. 9A, as shown in FIG. 10B.

The cock 28 is opened, and the stripping solution is infused into the bacteria collection unit 4C′ and is filtered through the filter 16. After that, an evaluation result can be obtained by same procedures as the procedures in the first modification.

The present modification is also capable of maintaining a substantially-sealed-system state and obtaining a reliable evaluation result as to cleanliness.

Although the example in which the base 29 including the cock 28 is provided in the collection container 26 has been described with reference to FIG. 10A, the cock 28 need not be provided. The stripping solution may be stored in (collected into) a syringe, and the stripping solution stored in the syringe may be infused into the bacteria collection unit 4C′ and be filtered through the filter 16, as shown in, e.g., FIG. 10B.

### Third Embodiment

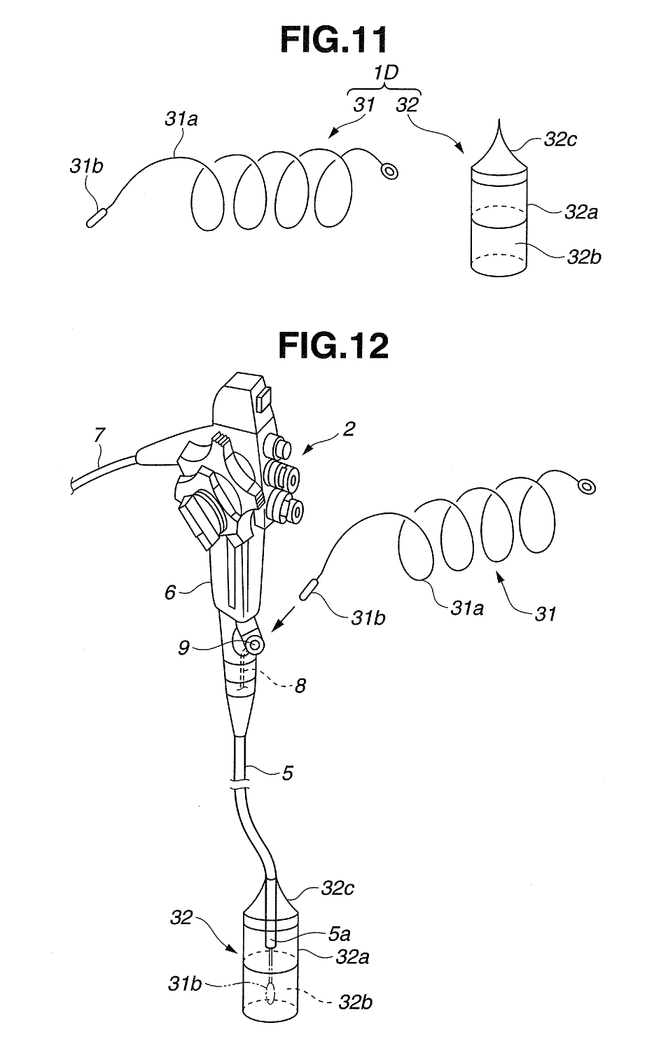

A third embodiment of the present invention will be described with reference to FIG. 11. FIG. 11 shows an evaluation apparatus ID according to the third embodiment. The evaluation apparatus ID has, e.g., a channel brush 31 with cotton which is inserted from an insertion opening 9 of an endoscope 2 into a channel 8 and harvests deposit from an inner surface of the channel 8 and a reagent-containing distal end cap 32 which seals a distal end side portion of an insertion portion 5. Note that the channel brush 31 with cotton and reagent-containing distal end cap 32 may be stored in a clean bag 18, as shown in FIG. 2, to form an evaluation kit for evaluation.

In the channel brush 31 with cotton, for example, a cotton portion 31b made of, e.g., capsular cotton is provided, at a distal end of a wire 31a, as a harvesting portion which harvests waste material adherent to the inner surface (inner wall) of the channel 8 by rubbing against the inner surface. For example, a ring-shaped portion to be grasped and operated by a user is provided at a rear end of the wire 31a.

Note that the cotton portion 31b as the harvesting portion is not limited to a portion using cotton, and a spongy material such as a sponge may be adopted instead. The cotton portion 31b is made of a member which can be readily impregnated with liquid and is elasticized.

In the reagent-containing distal end cap 32, a reagent solution 32b which detects protein in waste material is stored in, e.g., a cylindrical transparent container 32a. The reagent solution 32b is used for a biuret reaction and is composed of sodium hydrate and a copper sulfate solutions Note that a reagent which detects ATP instead of a reagent which detects protein may be used.

For example, a sealing film 32c similar to the sealing film 17 according to the first embodiment and the sealing member 27 according to the second modification of the second embodiment is provided at an upper end of the cylindrical transparent container 32a.

FIG. 12 shows how cleanliness of the channel 8 of the endoscope 2 is evaluated using the evaluation apparatus ID in FIG. 11.

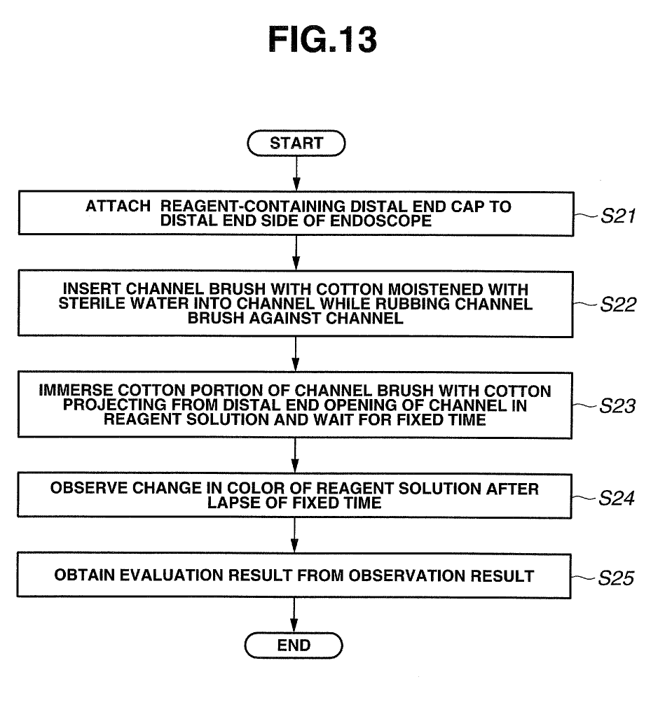

Procedures for evaluating cleanliness will be described with reference to FIG. 13.

In a first step, S21, a user attaches the reagent-containing distal end cap 32 to the distal end of the insertion portion 5 of the endoscope 2 in a sealed state from outside, as shown in FIG. 12. Alternatively, the user seals the distal end of the insertion portion 5 in the reagent-containing distal end cap 32.

In a next step, S22, the user moistens the cotton portion 31b at a distal end of the channel brush 31 with cotton with sterile water (or a sterilized stripping solution) and inserts the cotton portion 31b from the insertion opening 9 of the endoscope 2 toward a distal end opening of the channel 8 (more specifically a treatment instrument channel) while rubbing the cotton portion 31b against the inner surface of the channel 8.

Protein in waste material adherent to the inner surface of the channel 8 can be harvested more reliably by inserting the cotton portion 31b while rubbing the cotton portion 31b against the inner surface of the channel 8, as described above, than by inserting the cotton portion 31b without rubbing.

In a next step, S23, the user leaves the reagent solution 32b stand or waits for a fixed time (e.g., a reaction time required for a biuret reaction) while the cotton portion 31b of the channel brush 31 with cotton pushed out from the distal end opening is totally immersed in the reagent solution 32b.

FIG. 12 shows a state in which the distal end of the channel brush 31 with cotton (indicated by a chain double-dashed line), which has been inserted into the channel 8 of the endoscope 2 and is projecting from the distal end opening, is immersed in the reagent solution 32b. The reagent solution 32b is left stand for a reaction time required for protein detection by the reagent solution 32b, a biuret reaction time in the case.

In a next step, S24, the user observes a change in color of the reagent solution 32b after a lapse of the fixed time. To check in detail, transmittance or absorbance of light of a color (with a wavelength) corresponding to a biuret reaction may be measured using a spectroscope.

As shown in step S25, an evaluation result which is a result of simply quantifying protein adherent to the cotton portion 31b, i.e., left in the channel 8 (more specifically the treatment instrument channel) can be obtained on the basis of a result of the observation, i.e., the degree of change in color.

In this case, an evaluation result as to washing and disinfecting processing, i.e., cleanliness can be obtained by detecting presence or absence of protein in deposit on the inner surface of the channel 8 to check whether washing and disinfecting processing has been sufficiently performed.

More specifically, for example, if the reagent solution 32b has turned into purple, an evaluation result indicating insufficient washing and disinfecting processing can be obtained. On the other hand, if the reagent solution 32b has not turned into purple, an evaluation result indicating sufficient cleaning processing can be obtained.

According to the present embodiment, it is possible to simply evaluate protein or the like in waste material adherent to or left in the elongated channel 8 such as a channel of the endoscope 2 with high accuracy.

According to the present embodiment, it is possible to harvest more protein in waste material as deposit firmly adherent to the inner surface of the channel 8 by brushing with the cotton portion 31b than by simply supplying a stripping solution into the channel.

Since a reagent necessary for, e.g., a biuret reaction which reacts with waste material and changes in coloration is prepared in advance and is stored in the reagent-containing distal end cap 32, the trouble of reagent preparation is saved compared to a conventional example.

Note that although an example in which protein in waste material is detected has been described in the above explanation, the present embodiment can be applied to detection of ATP. The present embodiment can also be used in bacteria detection.

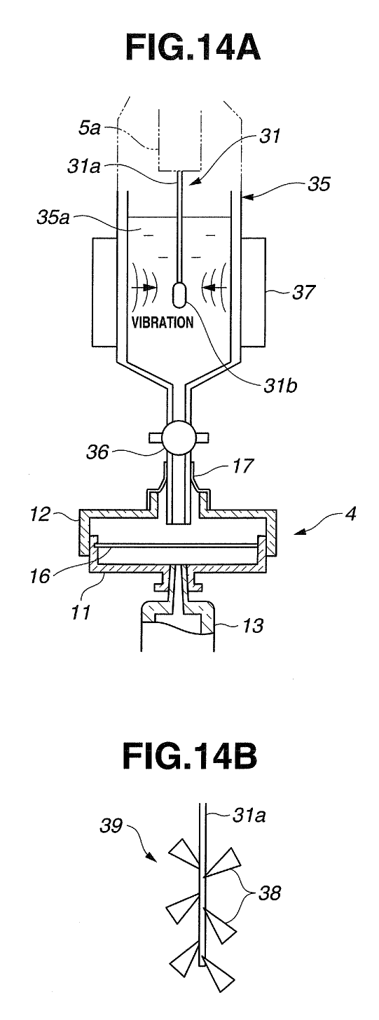

FIG. 14A shows a modification of the third embodiment. In the present modification, a container 35 containing a solution 35a such as sterile water or a (sterilized) stripping solution is adopted instead of the reagent-containing distal end cap 32 in FIG. 12.

Note that FIG. 14A shows a portion of the channel brush 31 with cotton in FIG. 12 which is projecting from a distal end portion 5a of the endoscope 2 on an enlarged scale. The container 35 is sealed from outside.

The cotton portion 31b of the channel brush 31 with cotton projecting from the distal end opening of the endoscope 2 is immersed in the solution 35a. In the present modification, a bacteria collection unit 4 provided with a filter 16 is attached at a bottom of the container 35 through a cock 36 which opens and closes.

In the present modification, a vibrator 37 which produces a vibration is detachably attached to a side surface of the container 35.

The vibrator 37 produces a vibration in the container 35. The vibration is transmitted to the cotton portion 31b inside the container 35. The transmission facilitates separation of bacteria adherent to the cotton portion 31b toward the solution 35a.

After the vibration is produced in the above-described manner, the cock 36 is opened, and the solution 35a is infused into the bacteria collection unit 4. At the time, the solution 35a is filtered through the filter 16. After that, an evaluation result can be obtained by same procedures as the procedures in the first embodiment.

Note that although an example in which the cotton portion 31b is provided at the distal end of the channel brush 31 has been described in the above explanation, for example, a channel brush 39 in which a plurality of spatulas 38 made of silicon rubber are provided at the distal end of the wire 31 a may be adopted, as shown in FIG. 14B. The case has substantially the same advantages as those of the case of the cotton portion 31b.

The reagent solution 32b may be filtered through the filter 16 of the bacteria collection unit according to the first or second embodiment, as the solution 35a. It is also possible to obtain an evaluation result as to washing and disinfecting processing by infusing a medium solution to reach the filter 16, through which the reagent solution 32b has been filtered, as described in the first or second embodiment and observing a surface of the medium solution after a lapse of a predetermined time.

### Fourth Embodiment

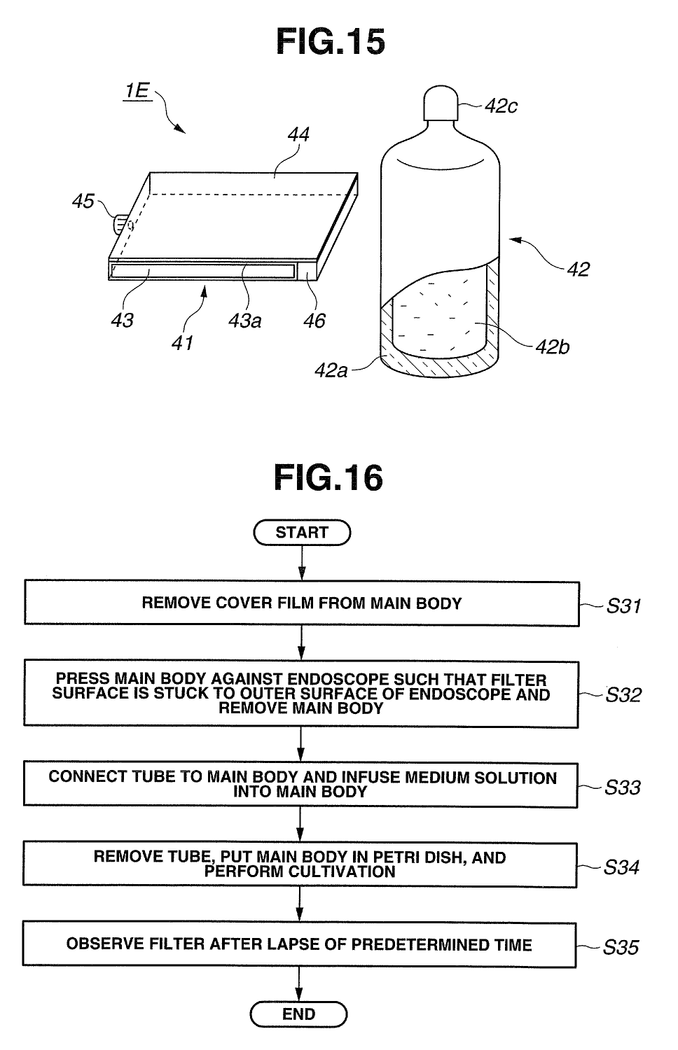

A fourth embodiment of the present invention will be described with reference to FIG. 15. FIG. 15 shows an evaluation apparatus 1E according to the fourth embodiment of the present invention. The first to third embodiments each relates to an apparatus which evaluates cleanliness of an interior of a channel. The present embodiment relates to an apparatus which evaluates cleanliness of, e.g., an outer surface of an endoscope 2.

The evaluation apparatus 1E includes a filter main body (hereinafter simply abbreviated as a main body) 41 used to harvest or collect bacteria directly from a portion to be evaluated and a medium tube (hereinafter simply abbreviated as a tube) 42 for supplying a medium solution to a filter having trapped bacteria harvested by the main body 41 and cultivating the bacteria.

The main body 41 includes, e.g., a sheet-like bag or storage portion 43, and one surface (an upper surface in the description below) forming part of the storage portion 43 is formed of a filter 43a.

A large number of holes having diameters of, e.g., 0.45 μm or less are formed in the filter 43a not to pass but to trap bacteria. Alternatively, the filter 43a is formed to be a net with a mesh size of 0.45 μm or less.

An upper surface of the filter 43a is initially in a sterilized state and is covered with a cover film 44 (note that a grasping portion 46 to be described below is also covered). The upper surface and an inner surface (a surface on the storage portion 43 side) of the filter 43a and an interior of the storage portion 43 are in an aseptic state.

In the storage portion 43, a base portion which is open to outside is covered with, e.g., a screw cap 45. The grasping portion 46 is provided on a side opposite to the base portion to make it easy for a user grasping the main body 41 to perform the work below.

In the tube 42, a sterilized medium solution 42b is stored in a tube main body 42a which is sterilized in advance, and a base which is open to outside is covered with a sterilized cap 42c.

Procedures for evaluating disinfecting processing of, e.g., an exterior portion of the endoscope 2 as a medical device to be evaluated according to the present embodiment will be described with reference to FIG. 16.

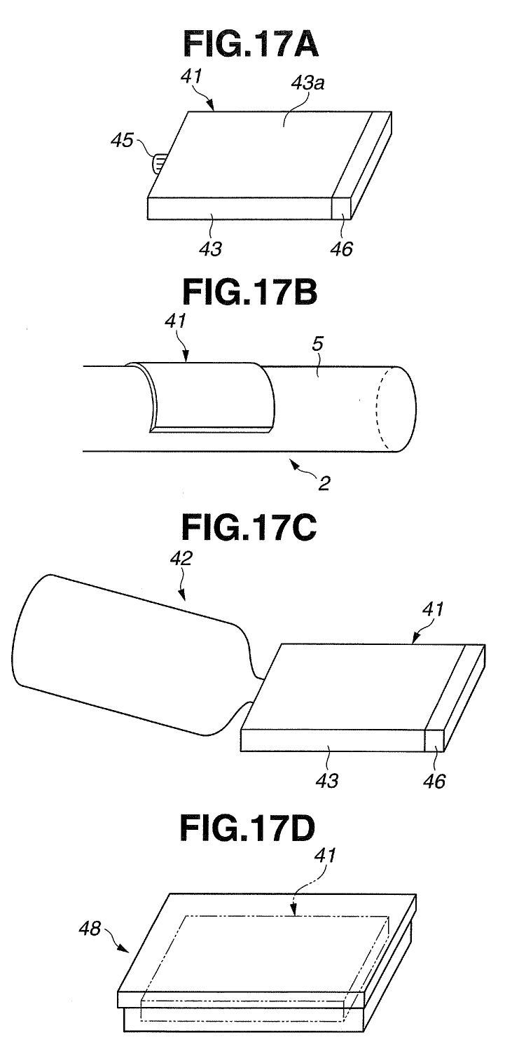

In a first step, S31, a user removes the cover film 44 from the main body 41. FIG. 17A shows the main body 41 with the cover film 44 removed.

In a next step, S32, the user pushes a clean surface of the filter 43a which is exposed after the cover film 44 is removed in the main body 41 against, e.g., an outer surface of an insertion portion 5 of the endoscope 2 to be evaluated such that the filter 43a is stuck to the outer surface. After that, the user removes the main body 41 from the endoscope 2 while grasping, e.g., the grasping portion 46. FIG. 17B shows a state in which the main body 41 is pressed against the outer surface of the insertion portion 5 of the endoscope 2.

The user removes the screw cap 45 of the main body 41 and also removes the cap 42c of the tube 42. In a next step, S33, the user connects the base of the tube 42 to the base portion of the main body 41 while inserting the base into the base portion and infuses the medium solution 42b in the tube 42 into the storage portion 43 of the main body 41 while manually squeezing the tube 42. FIG. 17C shows the infusion.

In the case, if the main body 41 is stored in a sterilized bag (not shown), and the infusion is performed in a sealed state, the surface of the filter 43a can be prevented from being contaminated. Rapid infusion of the medium solution 42b in the tube 42 into the storage portion 43 of the main body 41 reduces contamination by the air.

After infusing the medium solution 42b in the tube 42 into the storage portion 43 until the tube 42 becomes substantially empty, the user attaches the screw cap 45 to the main body 41.

In a next step, S34, the user puts the main body 41 in, e.g., a dedicated Petri dish 48 with the filter 43a on an upper surface of the main body 41 facing upward and performs cultivation at an appropriate temperature. FIG. 17D shows how the main body 41 is stored in the dedicated Petri dish 48.

As shown in step S35, the user observes a state of the filter 43a after a lapse of a predetermined time. More specifically, the user can obtain an evaluation result as to washing and disinfecting processing by measuring the number of bacteria cultivated in the filter 43a.

According to the present embodiment, an evaluation result as to washing and disinfecting processing can be obtained without troublesome operation. Evaluation can be performed without a clean room. Skilled aseptic operation is also unnecessary.

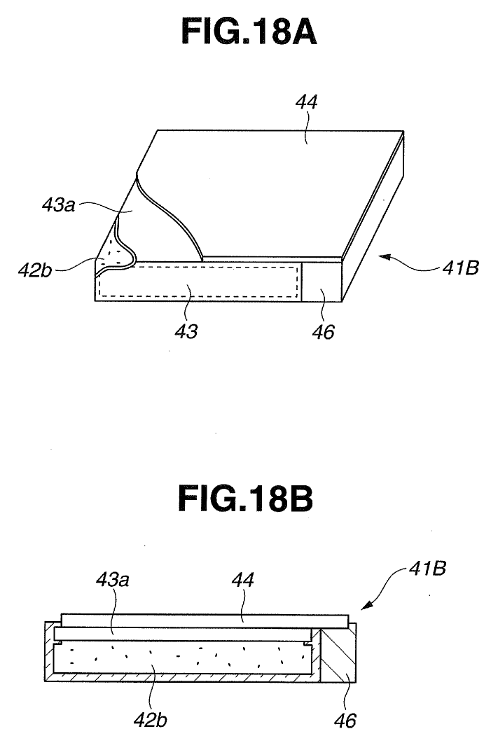

FIG. 18A shows a main body 41B according to a first modification. In the main body 41B of the first modification, the storage portion 43 is filled with the gel medium solution 42b in advance. A base portion through which the medium solution 42b is to be infused is thus unnecessary. FIG. 18B shows a cross section structure of the main body 41B.

A method of use is substantially the same as the method of use in the fourth embodiment. In the present case, the work of infusing the medium solution 42b is unnecessary. It is possible to prevent contamination by the air and obtain an evaluation result as to cleanliness.

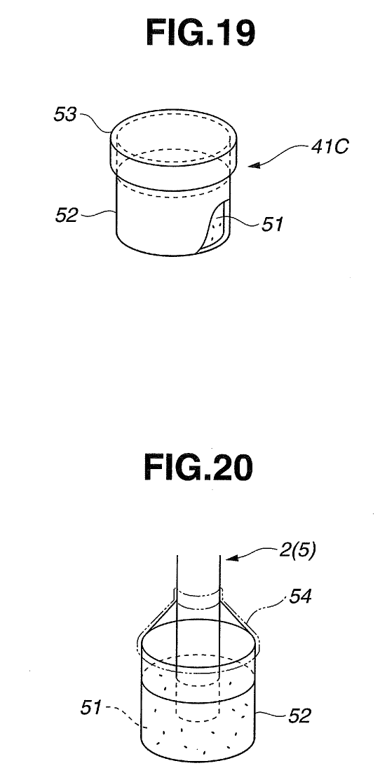

FIG. 19 shows an evaluation apparatus main body 41C according to a second modification. The evaluation apparatus main body 41C of the present modification is composed of a container 52 storing a solid medium 51 and a top cover 53 covering an open upper surface of the container 52. The present modification is suitable for checking (evaluating) disinfecting processing of a medical device portion which has a fine structure, such as a distal end surface of a distal end portion of an endoscope. Work in this case will be described below.

As shown in FIG. 20, the top cover 53 of the container 52 is removed, and a distal end surface of the insertion portion 5 of the endoscope 2 is pressed against or inserted into the solid medium 51 and is immediately removed. The container 52 is covered with the top cover 53 again, and cultivation is performed for a predetermined time. A result of the cultivation is observed. That is, bacteria are cultivated, and presence or absence or the approximate number of bacteria is measured. With the process, it is possible to check whether disinfecting processing of a medical device portion which has a fine structure, such as the distal end surface of the insertion portion 5, is sufficient.

Note that if the distal end surface of the endoscope 2 is pressed, as shown in FIG. 20, a sealing member 54 indicated by a chain double-dashed line may be attached to an upper surface of the container 52, thereby maintaining a sealed state when the distal end surface of the endoscope 2 is pressed against the solid medium 51 and preventing contamination by, e.g., bacteria in the air or the like.

An embodiment or the like obtained by, e.g., combining parts of the above embodiments and others also belongs to the present invention. The present invention can be applied to a medical device such as a treatment instrument including a channel, in addition to a channel of an endoscope.

According to the present invention, it is possible to obtain a reliable evaluation result as to cleanliness of a channel of an endoscope.

The present invention can be widely used to check whether a channel of an endoscope repeatedly used in endoscopic examinations has been sufficiently washed and disinfected.

Having described the embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope washing and disinfecting evaluation apparatus comprising:
a stripping solution storage section attached, in a state substantially sealed from outside, to one opening of a channel of an endoscope for storing a stripping solution to be infused from the one opening into the channel;
a bacteria collection container attached, in a state substantially sealed from outside, to the other opening of the channel in which a filter for trapping a bacterium contained in the stripping solution when the stripping solution having passed through the channel and flowed out from the other opening is filtered through the filter is placed; and
a medium supply section for placing the bacterium trapped by the filter in a state substantially sealed from outside and supplying a medium for cultivation.

2. The endoscope washing and disinfecting evaluation apparatus according to claim 1, wherein the stripping solution stored in the stripping solution storage section passes through the channel through suction operation by a suction section connected to an opening formed in the bacteria collection container in a state substantially sealed from outside and is filtered through the filter in the bacteria collection container.

3. The endoscope washing and disinfecting evaluation apparatus according to claim 1, wherein the stripping solution storage section comprises a stripping solution infusion section for infusing the stripping solution stored in the stripping solution storage section into the one opening, and the stripping solution infused from the one opening into the channel by the stripping solution infusion section passes through the channel and is filtered through the filter in the bacteria collection container.

4. The endoscope washing and disinfecting evaluation apparatus according to claim 1, wherein the filter is detachable from the bacteria collection container.

5. The endoscope washing and disinfecting evaluation apparatus according to claim 2, wherein the filter is detachable from the bacteria collection container.

6. The endoscope washing and disinfecting evaluation apparatus according to claim 3, wherein the filter is detachable from the bacteria collection container.

7. The endoscope washing and disinfecting evaluation apparatus according to claim 1, wherein the bacteria collection container is made of a transparent member which allows the bacteria collection container to be optically observed from outside.

8. The endoscope washing and disinfecting evaluation apparatus according to claim 2, wherein the bacteria collection container is made of a transparent member which allows the bacteria collection container to be optically observed from outside.

9. The endoscope washing and disinfecting evaluation apparatus according to claim 3, wherein the bacteria collection container is made of a transparent member which allows the bacteria collection container to be optically observed from outside.

10. The endoscope washing and disinfecting evaluation apparatus according to claim 4, wherein the bacteria collection container is made of a transparent member which allows the bacteria collection container to be optically observed from outside.

11. The endoscope washing and disinfecting evaluation apparatus according to claim 5, wherein the bacteria collection container is made of a transparent member which allows the bacteria collection container to be optically observed from outside.

12. The endoscope washing and disinfecting evaluation apparatus according to claim 6, wherein the bacteria collection container is made of a transparent member which allows the bacteria collection container to be optically observed from outside.

13. The endoscope washing and disinfecting evaluation apparatus according to claim 1, wherein the medium supply section is composed of a syringe which has an inlet attached to the bacteria collection container in a state substantially sealed from outside and infuses the medium into the bacteria collection container.

14. The endoscope washing and disinfecting evaluation apparatus according to claim 2, wherein the medium supply section is composed of a syringe which has an inlet attached to the bacteria collection container in a state substantially sealed from outside and infuses the medium into the bacteria collection container.

15. The endoscope washing and disinfecting evaluation apparatus according to claim 3, wherein the medium supply section is composed of a syringe which has an inlet attached to the bacteria collection container in a state substantially sealed from outside and infuses the medium into the bacteria collection container.

16. The endoscope washing and disinfecting evaluation apparatus according to claim 4, wherein the medium supply section is composed of a syringe which has an inlet attached to the bacteria collection container in a state substantially sealed from outside and infuses the medium into the bacteria collection container.

17. The endoscope washing and disinfecting evaluation apparatus according to claim 5, wherein the medium supply section is composed of a syringe which has an inlet attached to the bacteria collection container in a state substantially sealed from outside and infuses the medium into the bacteria collection container.

18. The endoscope washing and disinfecting evaluation apparatus according to claim 6, wherein the medium supply section is composed of a syringe which has an inlet attached to the bacteria collection container in a state substantially sealed from outside and infuses the medium into the bacteria collection container.

19. The endoscope washing and disinfecting evaluation apparatus according to claim 7, wherein the medium supply section is composed of a syringe which has an inlet attached to the bacteria collection container in a state substantially sealed from outside and infuses the medium into the bacteria collection container.

20. The endoscope washing and disinfecting evaluation apparatus according to claim 8, wherein the medium supply section is composed of a syringe which has an inlet attached to the bacteria collection container in a state substantially sealed from outside and infuses the medium into the bacteria collection container.

21. The endoscope washing and disinfecting evaluation apparatus according to claim 9, wherein the medium supply section is composed of a syringe which has an inlet attached to the bacteria collection container in a state substantially sealed from outside and infuses the medium into the bacteria collection container.

22. The endoscope washing and disinfecting evaluation apparatus according to claim 10, wherein the medium supply section is composed of a syringe which has an inlet attached to the bacteria collection container in a state substantially sealed from outside and infuses the medium into the bacteria collection container.

23. The endoscope washing and disinfecting evaluation apparatus according to claim 11, wherein the medium supply section is composed of a syringe which has an inlet attached to the bacteria collection container in a state substantially sealed from outside and infuses the medium into the bacteria collection container.

24. The endoscope washing and disinfecting evaluation apparatus according to claim 12, wherein the medium supply section is composed of a syringe which has an inlet attached to the bacteria collection container in a state substantially sealed from outside and infuses the medium into the bacteria collection container.

25. The endoscope washing and disinfecting evaluation apparatus according to claim 4, wherein the medium supply section is composed of a medium storage container storing a medium on which the filter having trapped a bacterium is placed.

26. The endoscope washing and disinfecting evaluation apparatus according to claim 5, wherein the medium supply section is composed of a medium storage container storing a medium on which the filter having trapped a bacterium is placed.

27. The endoscope washing and disinfecting evaluation apparatus according to claim 6, wherein the medium supply section is composed of a medium storage container storing a medium on which the filter having trapped a bacterium is placed.
